SEQUENCE LISTING (10 sequences)

SEQ ID NO: 1 - DNA, 254 bp, Agrostis stolonifera
catcaacaagcccaagatgcgccactacgtgcactgctacgcgctgcactgcctcgaccaggaggcctccgacgcgctgcgccgcgcgtacaaggcccgcggcgagaacgtcggcgcctggaggcaggcatgctacgcgccgctcgtcgacatcgccgccggccacggcttcgacgtcgacgccgtcttcgccgcgcacccgcgactcgccatctggtacgtgcccaccaggctccgccagctctgccaccagg

SEQ ID NO: 2 - DNA, 24 bp, Artificial
  primer
ctacatcaacaagcccaagatgcg

SEQ ID NO: 3 - DNA, 18 bp, Artificial
  primer
cctggtggcagagctggc

SEQ ID NO: 4 - DNA, 34 bp, Artificial
  Lox P site
ataacttcgtataatgtatgctatacgaagttat

SEQ ID NO: 5 - DNA, 1176 bp, Zea mays
atggatcccaacgacgccttctcggcggcgcacccgttccggtgggacctcggcccgccggcgcacgccgcgcccgcgcccgcgcctccgcctccgccgctagcaccgctgctgctgccgcctcacgcgccgcgggagctggaggacctggtggccggctacggcgtgcgcccgtccacggtggcgcggatctcggagctcgggttcacggcgagcacgctcctcggcatgacggagcgcgagctggacgacatgatggccgcgctcgcggggctgttccgctgggacgtgctcctcggcgagcgcttcggcctccgcgccgcgctgcgcgccgagcgcggccgcgtcatgtccctcggcgcccgctgcttccacgccgggagcaccttggatgccgcgtcacaagaagcgctgtccgacgagcgcgacgccgcggccagcggcggcggcatggcagaaggcgaggccggcaggaggatggtgacgacgaccgccggcaagaagggcaagaaaggggtcgttggcacgaggaagggcaagaaggcgaggaggaagaaggagctgaggccgctgaacgtgctggacgacgagaacgacggggacgagtacggcggcgggtcggagtcgaccgagtcgtccgcgggaggctccggggagaggcagcgggagcacccgttcgtggtcaccgagcccggcgaggtggcgagggccaagaagaacgggctcgactacctcttccacctgtacgagcagtgccgcgtcttcctgctccaggtgcagtccatcgctaagctgggcggccacaaatcccctaccaaggtgaccaaccaggtgttccggtacgcgaacaagtgcggggcgagctacatcaacaagcccaagatgcggcactacgtgcactgctacgcgctgcactgcctggacgaggaggcctccaacgcgctgcgccgggcgtacaagtcccgcggcgagaacgtgggcgcctggaggcaggcctgctacgcgccgctcgtcgagatcgccgcgcgccacggcttcgacattgacgccgtcttcgccgcgcacccgcgcctcgccgtctggtacgtgcccaccaggctgcgccagctctgccaccaggcgcgggggagccacgcccacgctgccgccggactcccgccgcccccgatgttctag

SEQ ID NO: 6 - DNA, 1185 bp, Zea mays
atggatcccaacgacgccttctcggcggcgcacccgttccggtgggacctgggcccgccggcccccgccgcgcccgcgcctccgcccccaccgccgcccgcgccgcagctgctgccccacgcgccgctgctgagcgcgccgagggagctggaggacctggtggccggctacggcgtgcgcccgtccacggtggcgcggatctcggagctcgggttcacggccagcacgctcctcggcatgacggagcgcgagctcgacgacatgatggccgcgctcgcggggctgttccgctgggacgtgctcctcggcgagcgcttcggcctccgcgccgcgctgcgggccgagcgcgggcgtgtcatgtccctcggcggccgcttccacaccgggagcacattggacgccgcgtcacaagaagtgctgtccgacgagcgcgacgccgcggccagcggcggcttagcggaaggcgaggccggcaggaggatggtgacgaccggcaagaagaagggcaagaaaggggttggcgcgaggaagggcaagaaggcgaggaggaagaaggagctgaggccgttggacgtgctggacgacgagaacgacggagacgaggacggcggcggcggcgggtcagactcgacggagtcttccgctggcggctccggcggcggggagaggcagcgggagcaccccttcgtggtcacagagcccggcgaggtggccagggccaagaagaacgggcttgactacctcttccatctgtacgagcagtgccgcgtcttcctgctgcaggtgcagtcccttgctaagctgggcggccacaagtcccctacaaaggtgaccaaccaggtgttccggtacgccaagaagtgcggcgcgagctacatcaacaagcccaagatgcggcactacgtgcactgctacgcgctgcactgcctggacgaggatgcctccaacgcgctgcgccgggcgtacaaggcccgtggcgagaacgtcggtgcctggaggcaggcctgctacgcgccgctcgtcgagatcgccgcgcgccacggcttcgacatcgacgccgtcttcgccgcgcacccgcgcctcaccatctggtacgtgcccaccaggttgcgccagctctgccaccaggcacgggggagccacgcccacgccgccgccggcctccccccgcccccgatgttctag

SEQ ID NO: 7 - DNA, 1170 bp, Oryza sativa
atggatcccaacgatgccttctcggccgcgcacccgttccggtgggacctcggcccgccggcgccggcgcccgtgccaccaccgccgccaccaccgccgccgccgccgccggctaacgtgcccagggagctggaggagctggtggcagggtacggcgtgcggatgtcgacggtggcgcggatctcggagctcgggttcacggcgagcacgctcctggccatgacggagcgcgagctcgacgacatgatggccgcgctcgccgggctgttccgctgggacctgctcctcggcgagcggttcggcctccgcgccgcgctgcgagccgagcgcggccgcctgatgtcgctcggcggccgccaccatgggcaccagtccgggagcaccgtggacggcgcctcccaggaagtgttgtccgacgagcatgacatggcggggagcggcggcatgggcgacgacgacaacggcaggaggatggtgaccggcaagaagcaggcgaagaagggatccgcggcgaggaagggcaagaaggcgaggaggaagaaggtggacgacctaaggctggacatgcaggaggacgagatggactgctgcgacgaggacggcggcggcgggtcggagtcgacggagtcgtcggccggcggcggcggcggggagcggcagagggagcatcctttcgtggtgacggagcccggcgaggtggcgagggccaagaagaacgggctggactacctgttccatctgtacgagcagtgccgcctcttcctgctgcaggtgcaatccatggctaagctgcatggacacaagtccccaaccaaggtgacgaaccaggtgttccggtacgcgaagaaggtcggggcgagctacatcaacaagcccaagatgcggcactacgtgcactgctacgcgctgcactgcctggacgaggaggcgtcggacgcgctgcggcgcgcctacaaggcccgcggcgagaacgtgggggcgtggaggcaggcctgctacgcgccgctcgtcgacatctccgcgcgccacggattcgacatcgacgccgtcttcgccgcgcacccgcgcctcgccatctggtacgtgcccaccagactccgccagctctgccaccaggcgcggagcagccacgccgccgccgccgccgcgctcccgccgcccttgttctaa

SEQ ID NO: 8 - DNA, 1203 bp, Lolium temulentum
atggatccccacgacgccttcctcgccgcgcacccgttccggtgggacctcggcccgccggctccggcggccgtgccccctcctcctccactgcccatgcctcaaactcccgcgctgcctccggcgaactcgccgagggagctggaggatctcgtggccgggtacggcgtgcgcggggccacggttgcgcgaatctccgagctcggcttcacggccagcacgctcctggtcatgacggaccgcgagctggacgacatgacggccgcactcgccggcctgttccgctgggacctgctcatcggcgagcggttcggcctgcgcgccgcgctgcgagcagagcgcggccgcctgatggcactgcatgggggccgacaccacggtcaccagtccggcagcaccatcgacggcgcctcccaagaagtgttgtccaacgaacgggatggggcggcgagcggcgaggacgacgccggcaggatgatgttatcgggcaagaagctgaagaatggatcggtggcgagaaaggccaagaaagcaaggaggaagaaggtggacgggctccggctggaccacatgcaggaggacgagcgcgaggacggcggcggccgctcggagtcaacggagtcgtcggctggcggaggcggcggcgttggaggggagcggcagcgggagcacccgttcgtggtgacggagcccggggaggtggcgagggccaagaagaacgggctggactacctgttccatctctacgagcagtgccgcctcttcctgctccaggtgcagtccatggccaagctgcatggccacaagtctccaaccaaggtgacgaaccaggtgttcaggtacgcgagcaaggtgggggcgagctacatcaacaagcccaagatgcgccactacgtgcactgctacgcgctgcactgcctcgaccaggaggcctccgacgcgctgcgccgcgcgtacaaggcccgcggcgagaacgtcggcgcctggaggcaggcatgctacgcgccgctcgtcgacatcgccgccggccacggcttcgacgtcgacgccgtcttcgccgcgcacccgcgactcgccatctggtacgtgcccaccaggctccgccagctctgccaccaggcaaggagcgcgcacgaagccgccgccgccaacgccaacgccaacggggccatgccgccgccgccgccgccgcccatgttctag

SEQ ID NO: 9 - DNA, 20 bp, Artificial
  primer
catcaacaagcccaagatgc

SEQ ID NO: 10 - DNA, 20 bp, Artificial
  primer
ggaggtgtacgggctctagg

(19) United States
(12) Patent Application Publication
(10) Pub. No.: US 2010/0122366 A1
(43) Pub. Date: May 13, 2010

(54) DEVELOPMENT OF CONTROLLED TOTAL VEGETATIVE GROWTH FOR PREVENTION OF TRANSGENE ESCAPE FROM GENETICALLY MODIFIED PLANTS AND FOR ENHANCING BIOMASS PRODUCTION

(75) Inventors: Hong Luo, Charlestown, RI (US); Joel M. Chandlee, South Kingstown, RI (US); Albert P. Kausch, Stonington, CT (US); Melvin J. Oliver, Lubbock, TX (US)

Correspondence Address: KLARQUIST SPARKMAN, LLP, 121 SW SALMON STREET, SUITE 1600, PORTLAND, OR, 97204, US

(73) Assignees: HybriGene, Inc.; Rhode Island Board of Governors for Higher Education

(21) Appl. No.: 12/573,806

(22) Filed: Oct. 5, 2009

Related U.S. Application Data
(63) Continuation of application No. 11/056,948, filed on Feb. 11, 2005, now abandoned.
(60) Provisional application No. 60/544,266, filed on Feb. 11, 2004.

(51) Int. Cl. A01H 5/00; C12N 15/82; A01H 1/02

(52) U.S. Cl. 800/260; 800/278; 800/298; 435/320.1

(57) ABSTRACT

Genes can be introduced into plants that confer desirable traits such as, drought and stress tolerance, insect and pest resistance, as well as environmental qualities such as phyto-remediation. However, possibility for transgene escape to wild and non-transformed species raises commercial and ecological concerns. Disclosed herein are methods and compositions for generating plants with total vegetative growth for the reduction, and in some examples prevention of, transgene escape. The same methods and compositions can also be used to increase biomass production in a plant.

### CROSS REFERENCE TO RELATED APPLICATION

This application is a continuation of U.S. patent application Ser. No. 11/056,948 filed Feb. 11, 2005, which claims the benefit of U.S. Provisional Application No. 60/544,266, filed Feb. 11, 2004, herein incorporated by reference.

### FIELD

This application relates to plant genome modification methods that result in controlled total floral sterile phenotypes and thus decrease transgene escape from a genetically modified plant and increase biomass production.

### BACKGROUND

The turfgrass industry includes many diverse groups, such as homeowners, athletic field managers, lawn care operators, golf course superintendents, architects, developers, landscape designers and contractors, seed and sod producers, parks and grounds superintendents, roadside and vegetation managers and cemetery managers. Turfgrass provides many environmental and societal benefits, including reducing soil erosion, filtering water, trapping dust and pollutants, reducing heat build-up in urban areas, and safer playing surfaces for athletes. The turfgrass seed market is only third to that of hybrid seed corn and soybeans. Therefore, trait improvement of turfgrass through genetic engineering is important to the turfgrass industry and the environment.

Beneficial traits such as herbicide resistance to reduce turfgrass management costs, drought and stress tolerance that will reduce water usage, insect and pest resistance that will cut pesticide applications, phyto-remediation of soil contaminants, and horticultural qualities such as aluminum tolerance, stay-green appearance, pigmentation and growth habit, can be improved in turfgrass. However, although turfgrass management and production is one of the fastest growing areas of agriculture, genetic transformation of turfgrasses lags behind that of many other important crop plants (Johnson and Riordan, 1999). The possibility of transgene escape from transgenic plants to wild and non-transformed species raises concerns regarding commercialization of transgenic turfgrass.

Although numerous risk assessment studies have been conducted on transgenic plants of annual and/or self-pollinating crops (Ellstrand and Hoffman, 1990; Hoffman, 1990; Dale, 1992; 1993; Rogers and Parkes, 1995; Ellstrand et al., 1999; Altieri, 2000; Dale et al., 2002; Eastham and Sweet, 2002), little information is available on the potential risks from the commercialization and large-scale seed production of perennial transgenic grasses. In a three-year field study on gene flow of transgenic bentgrass transformed with the bar gene (confers resistance to bialaphos and phosphinothricin-based herbicides), it was observed that pollen from the transgenic nursery traveled at least 411.5 feet, and that transgenes flowed to other species of Agrostis (Wipff and Friker, 2000; 2001). Recently, Watrud et al. (2004) conducted a landscape-level study on pollen-mediated gene flow from genetically modified creeping bentgrass (genetically engineered to contain the CP4 EPSPS gene that confers resistance to glyphosate), and observed multiple instances at numerous locations of long-distance viable pollen movement from multiple source fields of genetically modified creeping bentgrass.

Therefore, there is a need to develop methods that decrease, or even prevent transgene escape of transgenic plants into the environment. In flowering plants, gene flow can occur through movement of pollen grains and seeds. Various gene containment strategies have been developed to alter gene flow by interfering with flower pollination, fertilization, or fruit development (reviewed by Daniell, 2002). Interfering with the development of male reproductive structures through genetic engineering has been widely used as an effective strategy for the development of male sterility in plants. Selective ablation of tapetal cells by cell-specific expression of cytotoxic molecules (Moffatt and Somerville, 1988; Mariani et al., 1990; Tsuchiya et al., 1995; De Block et al., 1997; Jagannath et al., 2001) or an antisense gene essential for pollen development (Xu et al., 1995; Luo et al., 2000; Goetz et al., 2001) blocks pollen development, giving rise to male sterility. Male sterility, especially cytoplasmic male sterility (CMS) has been largely used in crop plants, such as rice and corn for the production of hybrid varieties. This strategy has been recently used in transgenic bentgrass for preventing transgene flow through pollen (Luo et al., 2004a; 2005a). Although male sterility appears to provide an effective method to control transgene flow in perennials, the development and evaluation of new strategies for gene containment in plant systems is needed. For example, the efficacy of male sterility in the prevention of transgene flow under the open-pollinated field conditions remains to be determined.

### SUMMARY

Disclosed herein are methods of reducing, and some examples preventing, transgene escape from a genetically modified transgenic plant by generating plants that grow substantially vegetatively. In some examples, such plants have increased biomass production, compared to a plant of the same species that is not genetically modified for substantial vegetative growth. In particular examples, the method produces total sterility in the plant. The implementation of controllable total vegetative growth in plants will not only reduce and in some examples eliminate the potential risks of transgene flow, but also facilitate the propagation and management of primary transgenics. Although particular examples are provided for controlling transgene escape in turfgrass, the disclosure is not limited to turfgrass. The methods disclosed can be applied to other transgenic plant species, such as those that can be propagated vegetatively, or to species, such as vegetables, for which seeds are not the final targeted products.

In particular examples, the method includes down-regulation of one or more plant genes that determine reproductive transition from a vegetative meristem, such as decreasing expression of one or more flower promotion genes, for example by using antisense or RNAi nucleic acid molecules of a flower promotion gene. In one example, this down regulation is controlled using a site-specific DNA recombination system to facilitate seed production (FIG. 1). In particular examples, down-regulation does not require 100% decrease in gene expression, but can include decreases of at least 50%, at least 75%, at least 90%, at least 95% or even at least 99%, for example as compared to an amount of gene expression in a non-transgenic plant.

In another example, the method includes up-regulation of one or more flower repressor genes, such as increasing expression of one or more flower repressor genes, for example by expressing a flower repressor cDNA in a plant, such as by operably linking a flower repressor cDNA (or fragment or variant thereof that retains at least 50% of the biological activity of the native sequence) to a constitutive or an inducible promoter. In some examples, up-regulation is controlled using a site-specific DNA recombination system. In particular examples, up-regulation includes increases of at least 20%, at least 50%, at least 75%, at least 90%, or even at least 100%, for example as compared to an amount of gene expression in a non-transgenic plant.

Because the disclosed methods increase vegetative growth, the disclosed methods can be used to enhance biomass production. For example, plants that grow vegetatively have an increase biomass production, compared to a plant of the same species that is not genetically modified for substantial vegetative growth. Examples of increases in biomass production include increases of at least 10%, at least 20%, or even at least 50%, when compared to an amount of biomass production by a plant of the same species not growing vegetatively.

In one example a method of reducing transgene escape by a transgenic plant, includes transforming a transgenic plant with a vector that promotes vegetative growth. For example, the vector can include a nucleic acid sequence that reduces expression of a flower promotion gene (such as an antisense or RNAi sequence that specifically recognizes a flower promotion gene). In another example, the vector can include a nucleic acid sequence that encodes a flower repressor gene (or functional variant or fragment thereof). The nucleic acid sequence is operably linked to a promoter, thereby producing a transgenic plant having total vegetative growth (such as significantly delayed flowering) and reducing transgene escape from the transgenic plant. In particular examples, the promoter is an inducible promoter, wherein expression of the nucleic acid sequence is achieved by exposing the plant to an agent that will induce the promoter. For example, if the promoter is a light-inducible promoter, the plant is exposed to light to “turn on” the inducible promoter and promote expression of the nucleic acid sequence operably linked to it.

In one example, the method includes crossing a first fertile transgenic plant having one or more desirable traits, with a second fertile transgenic plant, which can also have one or more desirable traits. For example, the first plant can be resistant to glufosinate and the second plant resistant to glyphosate. In particular examples, the first fertile plant contains a vector which includes a promoter operably linked to a blocking sequence (such as a selectable marker), wherein the blocking sequence is flanked by recombining site sequences. The vector also includes a sequence that interferes with (or decreases) expression of a flowering promotion gene (such as an antisense or RNAi of a flowering promotion sequence), or a sequence that increases expression of a flowering repressor gene sequence (such as a cDNA sequence), downstream of the promoter and blocking sequence, and positioned such that its expression is activated by the promoter in the presence of a recombinase, which results in recombination at the recombining site sequences and removal of the blocking sequence.

The second fertile plant can include another vector, wherein the vector includes a promoter, such as a constitutively active or inducible promoter, operably linked to a recombinase. If an inducible promoter is used, the second fertile plant is contacted with an inducing agent, before, during, or after crossing with the first fertile plant. The constitutively active promoter, or inducing agent that activates the inducible promoter, permits recombinase expression. The expressed recombinase protein interacts with the recombining sites of the other vector, resulting in recombination, removal of the blocking sequence such that the promoter is now operably linked to the nucleic acid sequence that reduces expression of a flower promotion gene, or to the nucleic acid sequence that increases expression of a flower repressor gene, thereby promoting expression of the nucleic acid sequence that reduces expression of a flower promotion gene, or expression of the flower repressor gene. The resulting progeny of this cross will have a total vegetative growth phenotype, and thus decreased transgene escape, and in some examples, increased biomass production.

In another example, instead of using two vectors, all of the elements are placed on a single vector, which is transformed into plants or plant cells. For example, the fertile plant can be transfected with a vector, wherein the vector includes a promoter (such as a constitutive promoter) operably linked to a blocking sequence. The blocking sequence is flanked by a recombining site sequence. The vector also includes a nucleic acid sequence that reduces expression of one or more flower promotion genes (such as an antisense or RNAi molecule of a flower promotion gene), or includes a nucleic acid sequence that increases expression of a flower repressor gene (such as a cDNA sequence of a flowering repressor gene). The nucleic acid sequence that reduces expression of a flower promotion gene or increases expression of a flower repressor gene is downstream of the blocking sequence, such that the nucleic acid sequence that reduces expression of a flower promotion gene or increases expression of a flower repressor gene is operably linked to the promoter upon recombination. The vector also contains an inducible promoter operably linked to a recombinase. The plant transformed with the vector is contacted with an inducing agent. The inducing agent activates the promoter, which promotes recombinase expression. The expressed recombinase interacts with the recombining sites, resulting in recombination, removal of the blocking sequence such that the promoter previously operably linked to a blocking sequence is now operably linked to the nucleic acid sequence that reduces expression of a flower promotion gene or to the nucleic acid sequence that increases expression of a flower repressor gene, thereby driving expression of the nucleic acid sequence that reduces expression of a flower promotion gene or increases expression of a flower repressor gene. The resulting plant has a total vegetative growth phenotype, and thus decreased transgene escape, and in some examples, increased biomass production.

In another example, the single vector containing all elements includes an inducible promoter operably linked to a nucleic acid sequence that reduces expression of a flower promotion gene or a nucleic acid sequence that increases expression of a flower repressor gene. The plant transformed with the vector is contacted with an inducing agent. The inducing agent activates the promoter, which promotes expression of the nucleic acid sequence that reduces expression of a flower promotion gene or the nucleic acid sequence that increases expression of a flower repressor gene. The resulting plant will have a total vegetative growth phenotype, and thus decreased transgene escape, and in some examples, increased biomass production.

Also provided by the present disclosure are plants produced by the disclosed methods, as well as seeds produced by the plants.

Also disclosed herein are vectors that can be used with the methods disclosed herein. For example, the disclosed vectors can include a promoter operably linked to a blocking sequence. The blocking sequence is flanked by a recombining site sequence, such as an FRT sequence, a lox sequence, a RS sequence, or a gix sequence. A particular example of a blocking sequence is a selectable marker nucleic acid sequence, such as a hyg, or bar, or pat gene sequence. The disclosed vectors can also include a sequence that disrupts expression of a flower promotion gene (such as an antisense or RNAi sequence that specifically recognizes a flower promotion sequence) downstream of the blocking sequence. Alternatively, the disclosed vectors can also include a flower repressor gene sequence downstream of the blocking sequence. In the presence of a recombinase, recombination of the recombining site sequences will remove the blocking sequence, result in the promoter being operably linked to the sequence that disrupts expression of a flower promotion gene or the flower repressor gene sequence.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram showing a particular example of a method that can be used to control total vegetative growth in plants. Transgenic plants containing a vector in which the rice ubiquitin promoter and an RNAi or antisense molecule of the flower-specific gene, FLO/LFY homolog, is separated by the hyg gene flanked by directly oriented FRT sites, will flower normally to produce seeds. However, when crossed to a plant expressing the FLP recombinase, FLP will excise the blocking fragment (hyg gene) thus bringing together the ubiquitin promoter and the downstream antisense (left) or RNAi construct (right) of the FLO/LFY homolog gene, turning off expression of the FLO/LFY homolog gene, resulting in total vegetative growth in the hybrid.

FIG. 2 is an alignment of cDNA sequences using CLUSTAL for FLO/FLY homologs in monocots (maize zfl1 (SEQ ID NO: 5) and zfl2: (SEQ ID NO:6); rice, RFL (SEQ ID NO: 7); and Lolium temulentum, LtLFY (SEQ ID NO: 8); SEQ ID NOS:5-8, respectively). Asterisks show conserved nucleotides and dashes indicate gaps to maximize alignment. Arrows indicate the primers designed for PCR amplification of the corresponding DNA fragment of bentgrass FLO/LFY homologs. Phylogentic tree derived from the sequence data using UPGMA method is also shown.

### SEQUENCE LISTING

The nucleic acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NO: 1 is a nucleic acid sequence showing the 3′ end of the FLO/LFY homolog gene in bentgrass used for RNAi and antisense constructs to transform bentgrass.

SEQ ID NOS: 2 and 3 are primers used to obtain the coding sequence of the 3′ end of the FLO/LFY homolog gene in bentgrass.

SEQ ID NO: 4 is a nucleic acid sequence showing a Lox P site.

SEQ ID NOS: 5-8 are cDNA sequences of maize (zfl1 and zfl2); rice (RFL); and Lolium temulentum (LtLFY) FLO/LFY homologs, respectively.

SEQ ID NOS: 9-10 are exemplary aligned nucleic acid sequences.

### DETAILED DESCRIPTION

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a floral meristem identity gene” includes a plurality of such genes and reference to “the vector” includes reference to one or more vectors and equivalents thereof known to those skilled in the art, and so forth. Similarly, the word “or” is intended to include “and” unless the context clearly indicates otherwise. Hence “comprising A or B” means including A, or B, or A and B.

Unless explained otherwise, all technical and scientific terms used herein have the same meaning as commonly understood to one of ordinary skill in the art to which this disclosure belongs.

Antisense: Nucleic acid molecules that are specifically hybridizable or specifically complementary to either RNA or the plus strand of a DNA sequence of interest, such as a flower-promotion DNA sequence. Antisense molecules can be used to interfere with or decrease gene expression, for example by at least 50% as compared to an amount of gene expression in the absence of the antisense molecule.

Biomass: The whole plant or green parts of a plant, such as leaves or vegetables. An increase in biomass production is an elevation in the amount or size of the plant, or green parts thereof, and can also include and increase in the nutrient content of the plant or its green parts.

Blocking sequence: Nucleic acid sequences located between two nucleic acid sequences of interest. Excision of a blocking sequence results in the two sequences being brought into operable association. For example, where the DNA sequence is located between a functional promoter and a nucleic acid sequence to be expressed from the promoter, excision of the blocking sequence results in the promoter and the nucleic acid sequence of interest being brought together to form a functional expression cassette. Exemplary blocking sequences include, but are not limited to, selectable markers, and those described in U.S. Pat. No. 5,925,808.

cDNA (complementary DNA): A piece of DNA lacking internal, non-coding segments (introns). cDNA can synthesized by reverse transcription from messenger RNA extracted from cells.

DNA (deoxyribonucleic acid): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Down-regulated or inactivation: When used in reference to the expression of a nucleic acid molecule, such as a gene, refers to any process which results in a decrease in production of a gene product. A gene product can be RNA (such as mRNA, rRNA, tRNA, and structural RNA) or protein. Therefore, gene down-regulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene down-regulation can include reduction of expression above an existing level. Examples of processes that decrease translation include those that decrease translational initiation, those that decrease translational elongation and those that decrease mRNA stability.

Gene down-regulation includes any detectable decrease in the production of a gene product. In certain examples, production of a gene product decreases by at least 2-fold, for example at least 3-fold or at least 4-fold, as compared to a control (such an amount of gene expression in a non-transgenic cell). In one example, a control is a relative amount of gene expression in a corresponding non-transgenic plant of the same variety of the transgenic plant.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule can be modulated compared to a normal (wild type) nucleic acid molecule. Modulation includes but is not limited to: (1) overexpression; (2) underexpression; or (3) suppression of expression. Modulation of the expression of a nucleic acid molecule can be associated with, and in fact cause, a modulation in expression of the corresponding protein.

Floral meristem identity (or floral initiation process) gene: A gene that determines (prevention or promotion) floral meristem identity upon the shoot apical meristem (SAM). Regulation of expression (up or down) of these genes can cause a SAM that develops into flowers in wild-type plants, to form structures with shoot-like characteristics. In one example, floral meristem identity genes activate the expression of organ identity genes that act later in flower development. Particular examples of such genes include, but are not limited to FLORICAULA (FLO) in Antirrhinum and its homolog LEAFY (LFY) in Arabidopsis; APETALA1/SQUAMOSA (AP1/SQUA) in Arabidopsis and Antirrhinum; CAULIFLOWER (CAL), FRUITFUL (FUL), FLOWERING LOCUS T (FLT), SUPPRESSOR OF OVEREXPRESSION OF CONSTANS1 (SOC1) in Aradopsis; TERMINAL FLOWER 1 (TFL1) in Arabidopsis and its homolog CENTRORADIALS (CEN) in Antirrhinum; FLOWERING LOCUS C (FLC) and EMF gene in Arabidopsis.

Flower-related gene: A gene that determines the transition from vegetative growth to reproductive phase of plant development. Particular examples include floral meristem identity genes (such as FLORICAULA (FLO) of Antirrhinum and its Arabidopsis counterpart LEAFY (LFY)), APETALA1/SQUAMOSA (AP1/SQUA) in Arabidopsis and Antirrhinum; CAULIFLOWER (CAL), FRUITFUL (FUL), FLOWERING LOCUS T (FLT), SUPPRESSOR OF OVEREXPRESSION OF CONSTANS1 (SOC1) in Aradopsis; TERMINAL FLOWER 1 (TFL1) in Arabidopsis and its homolog CENTRORADIALS (CEN) in Antirrhinum; FLOWERING LOCUS C (FLC) and EMF gene in Arabidopsis.

Flower (or flowering) promotion gene: A gene whose expression in a plant results in the development of flowers, or promotes transition into the reproductive phase of plant development. Examples include, but are not limited to: FLORICAULA (FLO) in Antirrhinum and its homolog LEAFY (LFY) in Arabidopsis; APETALA1 (Accession no. NM105581)/SQUAMOSA (AP1/SQUA) in Arabidopsis and Antirrhinum, CAULIFLOWER (CAL, Accession no. AY174609), FRUITFUL (FUL, Accession no. AY173056), FLOWERING LOCUS T (Accession no. AB027505), and SUPPRESSOR OF OVEREXPRESSION OF CONSTANS1 (SOC1) in Arabidopsis (Samach et al., 2000; Simpson and Dean, 2002; Zik and Irish, 2003).

Flower (or flowering) repressor gene: A gene whose expression disrupts the vegetative phase transition, or alters meristem identity. In particular examples, changing the timing or location of expression of a flower repressor gene can change the length of the vegetative phase length or flowering time. Examples include, but are not limited to: TERMINAL FLOWER 1 (TFL1, Accession no. NM120465) in Arabidopsis (Shannon and Meeks-Wagner, 1991) and its homolog CENTRORADIALS (CEN) in Antirrhinum (Bradley et al., 1996), FLOWERING LOCUS C (FLC; Michaels and Amasino, 1999; Accession no. AY769360) and EMF gene (Sung et al., 1992) in Arabidopsis.

Homolog: One sequence is homolog of another sequence, such as a gene, cDNA, or protein sequence, if the sequences share a particular amount of sequence identity, and have a similar biological function. In a particular example, homologs share at least 60% sequence identity, such as at least 70%, at least 80%, at least 90%, at least 95%, or even at least 99% sequence identity.

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Detects Sequences that Share at Least 80% Identity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share at Least 50% Identity)

| | |
|---|---|
| Hybridization: | 6x SSC at RT to 55° C. for 16-20 hours |
| Wash at least twice: | 2x-3x SSC at RT to 55° C. for 20-30 minutes each. |

Isolated: An “isolated” biological component (such as a nucleic acid molecule, protein, or organelle) has been substantially separated or purified away from other biological components in the cell of the organism in which the component naturally occurs, such as other chromosomal and extra-chromosomal DNA and RNA, proteins and organelles. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

Nucleic acid molecule: A deoxyribonucleotide or ribonucleotide polymer in either single or double stranded form, and unless otherwise limited, includes nucleic acid molecules that include analogues of natural nucleotides that can hybridize to nucleic acid molecules in a manner similar to naturally occurring nucleotides. In specific examples, nucleic acid molecules are linear or circular.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Promoter: An array of nucleic acid control sequences that directs transcription of a nucleic acid molecule. A promoter includes necessary nucleic acid sequences near the start site of transcription, such as a TATA element. A promoter also optionally includes distal enhancer or repressor elements which can be located as much as several thousand base pairs from the start site of transcription. Both constitutive and inducible promoters are included by this disclosure.

Specific, non-limiting examples of promoters include promoters derived from the genome of a plant cell (such as a ubiquitin promoter). Promoters produced by recombinant or synthetic techniques can also be used.

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis, by genetic engineering techniques, or other methods known in the art.

Recombinase: A protein which catalyses recombination of recombining sites. Particular examples of recombinases include, but are not limited to, a Cre protein, an Flp protein, a Tn3 recombinase, the recombinase of transposon gamma/delta, and the recombinase from transposon mariner.

The recombinases exert their effects by promoting recombination between two of their recombining sites. In the case of Cre, the recombining site is a Lox site, and in the case of Flp the recombining site is a Frt. Similar sites are found in transposon gamma/delta, TN3, and transposon mariner. Recombination between target sites arranged in parallel (so-called “direct repeats”) on the same linear DNA molecule results in excision of the intervening DNA sequence as a circular molecule. Recombination between direct repeats on a circular DNA molecule excises the intervening DNA and generates two circular molecules.

Recombining sites: Nucleic acid sequences that include inverted palindromes separated by an asymmetric sequence at which a site-specific recombination reaction can occur. In one specific, non-limiting example, a recombining site is a Lox P site (the target sequence recognized by the bacterial cre recombinase; such as the sequence ATAACTTCGTATAATGTATGCTA TACGAAGTTAT, SEQ ID NO: 4). In another specific non-limiting example, a recombining site is an FRT site. The FRT consists of two inverted 13-base-pair (bp) repeats and an 8-bp spacer that together comprise the minimal FRT site, plus an additional 13-bp repeat which may augment reactivity of the minimal substrate (for example see U.S. Pat. No. 5,654,182). In other, specific non-limiting examples, a recombining site is a recombining site from a TN3, a mariner, or a gamma/delta transposon.

RNA interference (RNAi): A post-transcriptional gene silencing mechanism mediated by double-stranded RNA (dsRNA). Introduction into cells of an RNAi gene construct whose expression results in the production in the targeted cell dsRNA (such as small interfering RNAs (siRNAs)), or direct introduction into cells of dsRNA, such as siRNAs or short hairpin RNAs (siRNAs) compounds, results in sequence-specific destruction of mRNAs, allowing targeted knockdown of gene expression. For example, a DNA molecule used for RNAi construction can be at least 100 base pairs (bp), at least 200 bp, or even at least 400 bp. In particular examples, the resulting RNAi molecule can be at least 20 nucleotides, at least 25 nucleotides, at least 30 nucleotides, or even at least 40 nucleotides, such as 20-40 nucleotides.

RNAi methods can be used to modulate transcription, for example, by decreasing or preventing gene expression, such as expression of a floral meristem identity gene. In certain examples, RNAi methods are designed to produce, in the targeted cells, siRNA molecules directed against a certain target gene, such as a bentgrass FLO/LFY homolog.

Selectable Marker: A sequence used to identify a cell of interest that expresses the sequence, such as expression of a nucleic acid sequence that results in production of a protein. A selectable marker can be detected using any method known to one of skill in the art, including enzymatic assays, spectrophotometric assays, antibiotic resistance assays, and assays utilizing antibodies (such as ELISA or immunohistochemistry). Specific non-limiting examples of selectable makers include enzymes (such as beta-galactosidase), fluorescent molecules (such as green fluorescent protein), antigenic epitopes, and antibiotic resistance proteins (such as proteins that provide resistance to zeomycin, hygromycin, tetracycline, puromycin or bleomycin).

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. To compare two nucleic acid sequences, the options can be set as follows: -i is set to a file containing the first nucleic acid sequence to be compared (such as C:\seq1.txt); -j is set to a file containing the second nucleic acid sequence to be compared (such as C:\seq2.txt); -p is set to blastn; -o is set to any desired file name (such as C:\output.txt); -q is set to −1; -r is set to 2; and all other options are left at their default setting. For example, the following command can be used to generate an output file containing a comparison between two sequences: C:\B12seq-i c:\seq1.txt-j c:\seq2.txt-p blastn-o c:\output.txt-q−1-r−2.

To compare two amino acid sequences, the options of B12seq can be set as follows: -i is set to a file containing the first amino acid sequence to be compared (such as C:\seq1.txt); -j is set to a file containing the second amino acid sequence to be compared (such as C:\seq2.txt); -p is set to blastp; -o is set to any desired file name (such as C:\output.txt); and all other options are left at their default setting. For example, the following command can be used to generate an output file containing a comparison between two amino acid sequences: C:\B12seq-i:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

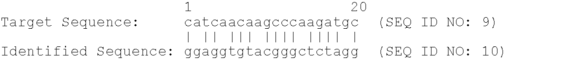

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Transformed: A transformed cell is a cell into which a nucleic acid molecule has been introduced, for example by molecular biology techniques. Transformation encompasses all techniques by which a nucleic acid molecule can be introduced into such a cell, including, but not limited to, Agrobacterium-mediated transformation, transfection with viral vectors, transformation with plasmid vectors, and introduction of nucleic acid molecules by electroporation, lipofection, and particle gun acceleration.

Transgene: A nucleic acid sequence that is exogenous to a cell. In one example, a transgene is a vector. In yet another example, the transgene is an RNAi or antisense nucleotide, wherein expression of the antisense or RNAi sequence decreases expression of a target nucleic acid sequence. A transgene can contain regulatory sequences, such as a promoter.

Transgenic plant: A plant that contains recombinant genetic material, for example nucleic acid sequences that are not normally found in plants of this type. In a particular example, a transgenic plant includes a vector that has been introduced by molecular biology methods. Includes a plant that is grown from a plant cell into which a recombinant nucleic acid was introduced by transformation, and all offspring of that plant that contain the introduced transgene (whether produced sexually or asexually).

Transgenic Cell: Transformed cells which contain foreign, non-native nucleic acid sequences, such as a vector.

Up-regulated or overexpression: When used in reference to the expression of a nucleic acid molecule, such as a gene, refers to any process which results in an increase in production of a gene product. A gene product can be RNA (such as mRNA, rRNA, tRNA, and structural RNA) or protein. Therefore, gene up-regulation or overexpression includes processes that increase transcription of a gene or translation of mRNA.

Gene up-regulation includes any detectable increase in the production of a gene product. In certain examples, production of a gene product increases by at least 20%, at least 50%, or even at least 100%, as compared to a control (such an amount of gene expression in a non-transgenic cell). In one example, a control is a relative amount of gene expression in a corresponding non-transgenic plant of the same variety of the transgenic plant.

Variants of Amino Acid and Nucleic Acid Sequences: The production of the disclosed vectors can be accomplished in a variety of ways. One of ordinary skill in the art will appreciate that a DNA sequence can be altered in numerous ways without affecting the biological activity of DNA sequences. For example, PCR can be used to produce variations in the DNA sequence of a vector. In one example, a variant sequence is optimized for expression.

In one example, a variant is a sequence change to a cDNA sequence. Two types of cDNA sequence variant can be produced. In the first type, the variation in the cDNA sequence is not manifested as a change in the amino acid sequence of the encoded peptide. These silent variations reflect the degeneracy of the genetic code. In the second type, the cDNA sequence variation changes the amino acid sequence of the encoded protein. In such cases, the variant cDNA sequence produces a variant peptide sequence. In order to optimize preservation of the functional and immunologic identity of the encoded polypeptide, any such amino acid substitutions can be conservative. Conservative substitutions replace one amino acid with another amino acid that is similar in size, hydrophobicity, and so forth. Such substitutions generally are conservative when it is desired to finely modulate the characteristics of the protein. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Variations in the cDNA sequence that result in amino acid changes, whether conservative or not, are minimized to enhance preservation of the functional and immunologic identity of the encoded protein. In particular examples, a cDNA sequence variant will introduce no more than 20, for example fewer than 10 amino acid substitutions into the encoded polypeptide, such as 1-10 amino acid substitutions. Variant amino acid sequences can, for example, be 80%, 90% or even 95% identical to the native amino acid sequence.

Conserved residues in the same or similar proteins from different species can also provide guidance about possible locations for making substitutions in the sequence. A residue which is highly conserved across several species is more likely to be important to the function of the protein than a residue that is less conserved across several species.

Vector: A nucleic acid molecule as introduced into a cell, such as a plant cell, thereby producing a transformed cell. A vector can include nucleic acid sequences that permit it to replicate in a host cell, such as an origin of replication. A vector can also include one or more selectable marker genes, such as an antibiotic resistance marker, and other genetic elements known in the art.

Vegetative growth: The life cycle of flowering plants in general can be divided into three growth phases: vegetative, inflorescence, and floral. In the vegetative phase, the shoot apical meristem (SAM) generates leaves that later will ensure the resources necessary to produce fertile offspring. Upon receiving the appropriate environmental and developmental signals the plant switches to floral, or reproductive, growth and the SAM enters the inflorescence phase (I1) and gives rise to an inflorescence with flower primordia. During this phase the fate of the SAM and the secondary shoots that arise in the axils of the leaves is determined by a set of meristem identity genes, some of which prevent and some of which promote the development of floral meristems. Once established, the plant enters the late inflorescence phase (I2) where the floral organs are produced. If the appropriate environmental and developmental signals the plant switches to floral, or reproductive, growth are disrupted, the plant will not be able to enter reproductive growth, therefore maintaining vegetative growth.

The term “total vegetative growth” includes plants that do not enter the reproductive growth stage, and in some examples includes plants having a significant delay in flowering, such as a delay of at least one month, at least two months, at least three months, or even at least six months.

Methods of Reducing Transgene Escape and Increasing Biomass Production

Methods for reducing, such as preventing, transgene escape from a genetically modified (transgenic) plant are disclosed. In particular examples, such methods can also be used to increase biomass production of a plant. In some examples, the resulting reduced transgene escape or enhanced biomass production is maintained through vegetative propagation of the plant. The methods include changing expression of (such as down-regulating or up-regulating) a flower-related gene, wherein the change in expression results in total vegetative growth of the transgenic plant. For example, expression of a flower-promotion gene can be down-regulated, and expression of a flower repressor gene can be up-regulated, to promote vegetative growth, thereby decreasing transgene escape, and in some examples increasing biomass production. The methods can include using FLP-mediated site-specific DNA excisional recombination for controlled vegetative growth.

The disclosed methods can further include selecting those transgenic or hybrid progeny resulting from a cross, that have decreased trangene escape or increased biomass production.

The disclosed methods are not limited to reducing transgene escape and increasing biomass production in particular species of plants. Although particular examples are provided for reducing transgene escape in turfgrass, the methods of the present disclosure can be used in annuals and perennials (such as turfgrass), and can be used to reduce transgene escape in monocots (such as rice, maize and forage grasses) and dicots (such as Antirrhinum and Arabidopsis).

The disclosed methods can be used to decrease transgene escape (and in some examples also increase biomass production) in a transgenic plant having one or more desirable traits. Exemplary desirable traits include, but are not limited to, herbicide resistance, drought tolerance, salt tolerance, and disease resistance. In particular examples, a desirable trait is linked to decreased transgene escape or increased biomass production.

Also provided by the present disclosure are plants produced using the methods disclosed herein, as well as seeds from such plants. For example transgenic plants having total vegetative growth (such as no flower production or a significant delay in flowering), as well as transgenic plants having enhanced biomass production, are provided by the present disclosure.

### Flower-Related Genes

The life cycle of flowering plants is generally divided into three growth phases: vegetative, inflorescence, and floral. The switch from vegetative to reproductive development requires a change in the developmental program of the descendents of the stem cells in the shoot apical meristem (SAM). In the vegetative phase the SAM generates leaves that provide resources necessary to produce fertile offspring. Upon receiving the appropriate environmental and developmental signals the plant switches to floral, or reproductive, growth and the SAM enters the inflorescence phase (I1) and gives rise to an inflorescence with flower primordia. During this phase the fate of the SAM and the secondary shoots that arise in the axils of the leaves is determined by a set of meristem identity genes, some of which prevent and some of which promote the development of floral meristems. Once established, the plant enters the late inflorescence phase (I2) where the floral organs are produced. Two basic types of inflorescences have been identified in plants: determinate and indeterminate. In determinate species the SAM eventually produces floral organs and the production of meristems is terminated with a flower. The SAM of indeterminate species is not converted to a floral identity and will therefore only produce floral meristems from its periphery, resulting in a continuous growth pattern.

In dicots, after the transition from vegetative to reproductive development, floral meristems are initiated by the action of a set of genes called floral meristem identity genes. Among them, FLORICAULA (FLO) of Antirrhinum and its Arabidopsis counterpart LEAFY (LFY) participate in the reproductive transition to establish floral fate. In strong flo and lfy mutant plants, flowers are transformed into inflorescence shoots (Coen et al., 1990; Weigel et al., 1992), indicating that FLO and LFY are exemplary flower-promotion genes. It is hypothesized that FLO/LFY are responsible for the initial steps in flower initiation.

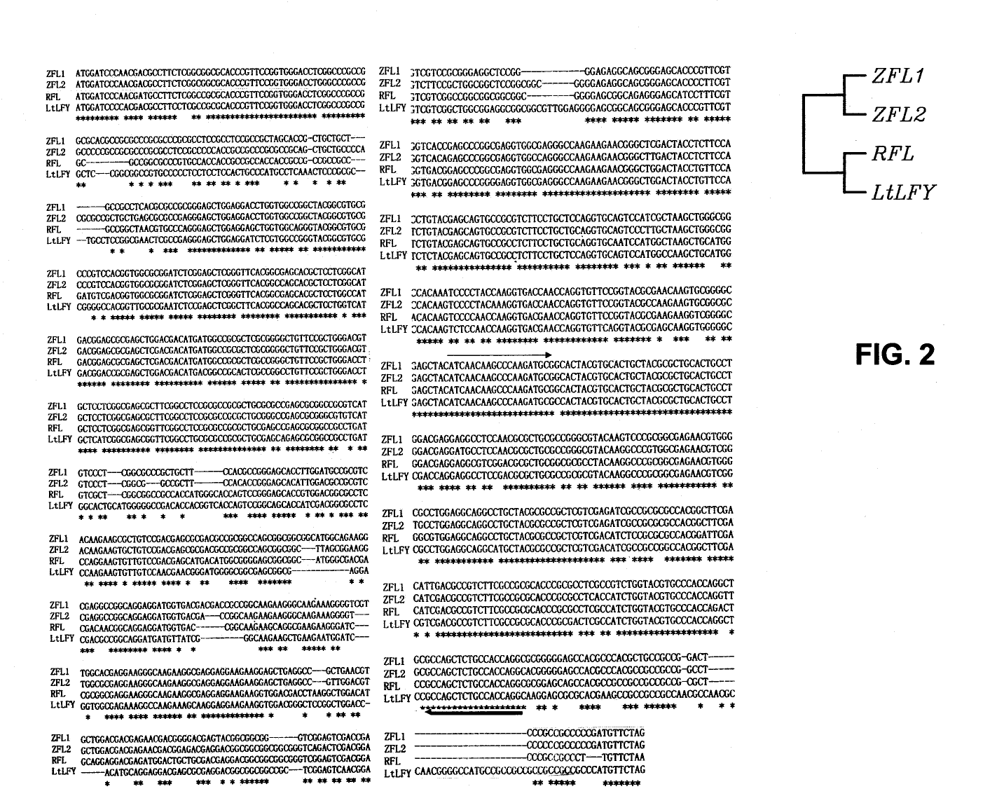

In monocots, FLO/LFY homologs have been identified in several species, such as rice (Kyozuka et al., 1998); Lolium temulentum, maize, and ryegrass (Lolium perenne) whose FLO/LFY homologs are almost identical at the nucleotide level. The FLO/LFY homologs from different species have high homology in amino acid sequences, and are well conserved in the C-terminal regions (Kyozuka et al., 1998; Bomblies et al., 2003). This has also been observed at DNA level (FIG. 2). It appears that the function of the FLO/LFY homologs in controlling the switch from vegetative to reproductive development is conserved among different species.

In addition to FLO/LFY genes, reduced expression of other flower-promotion genes that promote the transition from vegetative growth to reproductive growth can result in vegetative growth of the transgenic plant, thus decreasing or preventing transgene escape. In particular examples, such a method also increases biomass production of the transgenic plant. Additional examples of flower promotion genes include, but are not limited to: APETALA1 (Accession no. NM105581)/SQUAMOSA (AP1/SQUA) in Arabidopsis and Antirrhinum, CAULIFLOWER (CAL, Accession no. AY174609), FRUITFUL (FUL, Accession no. AY173056), FLOWERING LOCUS T (Accession no. AB027505), and SUPPRESSOR OF OVEREXPRESSION OF CONSTANS1 (SOC1) in Arabidopsis (Samach et al., 2000; Simpson and Dean, 2002; Zik and Irish, 2003).

In particular examples, down-regulation of expression of one or more flower promotion genes in a plant, such as a FLO/LFY homolog, will result in total vegetative growth in the transgenic plant, whereby the transgenic plant is unable to produce flowers (or there is a significant delay in flower production). Because FLO/LFY homologs have high homology, additional FLO/LFY homologs can be isolated from other species, for example bentgrass, for example by using the methods of Kyozuka et al., 1998 and Bomblies et al., 2003. The 3′-end of the bentgrass (Agrostis stolonifera L.) FLO/LFY homolog has been cloned (SEQ ID NO: 1). The vegetatively grown transgenic plants can reduce transgene escape through a reproductive pathway, such as a reduction of at least 10%, at least 20%, at least 50%, at least 75%, at least 90%, or even at least 95%, for example relative to a transgenic plant not down-regulated for expression of one or more flower promotion genes. In particular examples, the vegetative growth will increase biomass production of the plant of interest, such as an increase of at least 10%, at least 20%, at least 50%, at least 75%, at least 90%, or even at least 95%, for example relative to a transgenic plant not down-regulated for expression of one or more flower promotion genes. Any method known in the art can be used to reduce or down-regulate expression of a FLO/LFY homolog or other flower promotion gene in a plant. In particular examples, antisense or RNAi approaches are used.

In particular examples, down-regulation of expression of a flower promotion gene does not require a 100% reduction in such expression. For example, a reduction of at least 50%, at least 75%, at least 95%, or even at least 99%, as compared to expression of the gene in a non-transgenic plant of the same species, indicates that expression of the gene was down regulated. In particular examples, down-regulation reduces expression by 100%, such that expression of the gene is not detectable.

As an alternative to down-regulating expression of one or more flower-promotion genes to prevent flower development, expression of one or more flower-repressor genes can be up-regulated using methods known in the art. Flower repressor genes can disrupt the vegetative phase transition or alter meristem identity. Particular examples of such genes include, but not limited to: TERMINAL FLOWER 1 (TFL1, Accession no. NM120465) in Arabidopsis and its homolog CENTRORADIALS (CEN) in Antirrhinum (Bradley et al., 1996), FLOWERING LOCUS C (FLC, Accession no. AY769360) and EMF (Sung et al., 1992) in Arabidopsis.

Increased expression of a flower-repressor gene can result in vegetative growth of the transgenic plant, thus decreasing transgene escape. For example, overexpression of one or more flower repressor genes in a plant will result in delay or suppression of flowering in the transgenic plant, an in some examples an inability to produce flowers. As described above, the vegetatively grown transgenic plants can reduce transgene escape through a reproductive pathway, such as a reduction of at least 10%, at least 20%, at least 50%, at least 75%, at least 90%, or even at least 95%, for example relative to a transgenic plant not up-regulated for expression of one or more flower repressor genes. In particular examples, the vegetative growth will increase biomass production of the plant of interest, such as an increase of at least 10%, at least 20%, at least 50%, at least 75%, at least 90%, or even at least 95%, for example relative to a transgenic plant not up-regulated for expression of one or more flower repressor genes.

### Promoters

Any method known in the art can be used to increase or up-regulate expression of a flower repressor gene in a plant, or to decrease or down-regulate expression of a flower promoter gene in a plant. In particular examples, a cDNA encoding the desired flower repressor protein (or fragment or variant thereof having at least 50% of the biological activity of the native sequence), or an RNAi or antisense molecule that specifically recognizes a flower promoter gene, is expressed under the control of a promoter. For example, constitutive and flower-specific promoters can be used to promote gene expression. Constitutive promoters function under most environmental conditions. Any constitutive promoter, including variants thereof that are functionally equivalent and confer gene expression in plant tissues and cells, can be used to express a nucleic acid sequence, such as a cDNA, RNAi, or antisense sequence, in a transgenic plant. Exemplary constitutive promoters include, but are not limited to, promoters from plant viruses such as the 35S promoter from CaMV (Odell et al., Nature 313:810-2, 1985; U.S. Pat. No. 5,858,742 to Fraley et al.); promoters from plant genes as rice actin (McElroy et al., Plant Cell 2:163-71, 1990); ubiquitin (Christensen et al., Plant Mol. Biol. 12: 619-32, 1989); pEMU (Last et al., Theor. Appl. Genet. 81:581-8, 1991); MAS (Velten et al., EMBO J. 3:2723-30, 1984); maize H3 histone (Lepetit et al., Mol. Gen. Genet. 231:276-85, 1992 and Atanassova et al., Plant J. 2:291-300, 1992); and the ALS promoter, a XbaI/NcoI fragment 5′ to the Brassica napus ALS3 structural gene or a nucleotide sequence with substantial sequence similarity (PCT Application No. WO 96/30530). A particular example is a rice ubiquitin gene promoter (Genbank accession no. AF184280).

In another example, the promoter used is an inducible promoter, such as a promoter responsive to environmental stimuli or synthetic chemical. Exemplary inducible promoters include those induced by heat, a chemical, or light. Use of an inducible promoter allows for controlling total vegetative growth. For example, the use of an inducible promoter permits normal expression of the flowering promotion genes, such as FLO/LFY-homologs, in transgenic plants during seed multiplication, and then down-regulation of flowering promotion genes when total vegetative growth is desired. Alternatively, expression of one or more flowering repressor genes can be reduced or down-regulated in transgenic plants during seed multiplication, and then allowed to be expressed to permit total vegetative growth (for example when grown under non-controlled field conditions).

### RNAi Construction

RNAi constructs can be used to decrease or inhibit expression of any flower promotion sequence, such as a FLO/LFY-homolog. One skilled in the art will understand that RNAi constructs can be generated to flower promotion gene. In particular examples, an RNAi construct includes a DNA sequence that is a portion of a target gene, arranged in sense and antisense orientations under the control of a promoter. The transcription of the sense and the antisense DNA sequence results in a dsRNA, then siRNA. The siRNA molecule can cause sequence-specific destruction of mRNAs, allowing targeted knockdown of gene expression. In one example, a DNA sequence used for an RNAi construct is specific for SEQ ID NO: 1. This disclosure is not limited to RNAi compounds of a particular length. A DNA sequence used for an RNAi construct can be any length, such as at least 100 bp, at least 200 bp, at least 300 bp, or even at least 400 bp.

For example, a 200 bpDNA sequence can be used to generate an RNAi construct. In particular examples, this RNAi construct is introduced into a plant cell, such as a cell of a plant in which decreased transgene escape or increased biomass production is desired. Such methods will result in production of an siRNA molecule that will decrease expression, such as expression of a flower-related gene.

### Antisense Nucleic Acid Molecules

One approach to disrupting flower promotion function or expression is to use antisense oligonucleotides. To design antisense oligonucleotides, a flower promotion mRNA sequence, such as a floral meristem sequence, is examined. Regions of the sequence containing multiple repeats, such as TTTTTTTT, are not as desirable because they will lack specificity. Several different regions can be chosen. Of those, oligos are selected by the following characteristics: those having the best conformation in solution; those optimized for hybridization characteristics; and those having less potential to form secondary structures. Antisense molecules having a propensity to generate secondary structures are less desirable.

Plasmids or vectors including the antisense sequences of a flower promotion sequence can be generated. For example, cDNA fragments or variants coding for a flower promotion protein can be PCR amplified and cloned in antisense orientation in a vector. The nucleotide sequence and orientation of the insert can be confirmed by sequencing using a Sequenase kit (Amersham Pharmacia Biotech).

Generally, the term “antisense” refers to a nucleic acid molecule capable of hybridizing to a portion of a flower promotion RNA (such as mRNA) by virtue of some sequence complementarity. The antisense nucleic acid molecules disclosed herein can be oligonucleotides that are double-stranded or single-stranded, RNA or DNA or a modification or derivative thereof, which can be incorporated into a vector and transfected into a plant or plant cell, to permit expression of the antisense sequence in the cell.

Flower promotion antisense nucleic acid molecules are polynucleotides, and can include sequences that are at least 6 by in length. In particular examples, antisense sequences range from about 6 to about 500 by in length, such as 6-100 bp. A flower promotion antisense polynucleotide recognizes any species of a flower promotion gene sequence. In specific examples, the polynucleotide is at least 10, at least 15, at least 100, at least 200, or at least 500 bp. However, antisense nucleic acid molecules can be much longer. The nucleotides of the antisense sequence can be modified at the base moiety, sugar moiety, or phosphate backbone, and can include other appending groups such as peptides, or agents facilitating transport across the cell membrane.

A flower promotion antisense polynucleotide, such as a single-stranded DNA, can be modified at any position on its structure with substituents generally known in the art. For example, a modified base moiety can be 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, and 2,6-diaminopurine.

In another example, a flower promotion antisense molecule includes at least one modified sugar moiety such as arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In yet another example, a flower promotion antisense molecule is an α-anomeric oligonucleotide. An α-anomeric oligonucleotide forms specific double-stranded hybrids with complementary RNA in which, contrary to the usual 13-units, the strands run parallel to each other (Gautier et al., Nucl. Acids Res. 15:6625-41, 1987). The oligonucleotide can be conjugated to another molecule (such as a peptide, hybridization triggered cross-linking agent, transport agent, or hybridization-triggered cleavage agent). Oligonucleotides can include a targeting moiety that enhances uptake of the molecule by cells. The targeting moiety can be a specific binding molecule, such as an antibody or fragment thereof that recognizes a molecule present on the surface of the cell, such as a plant cell.

Antisense molecules can be synthesized by standard methods, for example by use of an automated DNA synthesizer. As examples, phosphorothioate oligos can be synthesized by the method of Stein et al. (Nucl. Acids Res. 1998, 16:3209), methylphosphonate oligos can be prepared by use of controlled pore glass polymer supports (Sarin et al., Proc. Natl. Acad. Sci. USA 85:7448-51, 1988). In a specific example, an antisense oligonucleotide that recognizes a flower promotion sequence includes catalytic RNA, or a ribozyme (see WO 90/11364, Sarver et al., Science 247:1222-5, 1990). In another example, the oligonucleotide is a 2′-0-methylribonucleotide (Inoue et al., Nucl. Acids Res. 15:6131-48, 1987), or a chimeric RNA-DNA analogue (Inoue et al., FEBS Lett. 215:327-30, 1987).

The antisense nucleic acids disclosed herein include a sequence complementary to at least a portion of an RNA transcript of a flower promotion gene. However, absolute complementarity, although advantageous, is not required. A sequence can be complementary to at least a portion of an RNA; in the case of double-stranded antisense nucleic acids, a single strand of the duplex DNA can thus be tested, or triplex formation can be assayed. The ability to hybridize depends on the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The relative ability of polynucleotides to bind to complementary strands is compared by determining the Tₘ, of a hybridization complex of the poly/oligonucleotide and its complementary strand. The higher the Tₘ, the greater the strength of the binding of the hybridized strands. As close to optimal fidelity of base pairing as possible achieves optimal hybridization of an oligonucleotide to its target RNA.

### Site-Specific DNA Recombination

Site-specific DNA recombination can be used to produce transgenic plants that have reduced transgene escape. Site-specific recombination is a process involving reciprocal exchange between specific DNA recombining sites catalyzed by recombinases. Site-specific recombinases recognize specific DNA sequences, and in the presence of two such recombination sites, catalyze the recombination of DNA strands. Recombinases can catalyze excision or inversion of a DNA fragment according to the orientation of their specific target sites. Recombination between directly oriented sites leads to excision of the DNA between them, whereas recombination between inverted target sites causes inversion of the DNA between them. Some site-specific recombination systems do not require additional factors for their function and are capable of functioning accurately and efficiently in various heterologous organisms.

One particular example of a site-specific recombination system is the Cre/lox system of bacteriophage P1. Cre recombinase can excise, invert, or integrate extrachromosomal DNA molecules in plant cells. Another particular example of a site-specific recombination system is the FLP/FRT recombination system of yeast. The recombinase FLP can catalyze efficient recombination reactions in heterologous eukaryotic cells For example, Lyznik et al. (1993) used a modified FLP coding sequence from pOG44 (O'Gorman et al., 1991) to synthesize a chimeric plant FLP gene driven by the maize ubiquitin promoter to show activity of FLP recombinase in maize and rice cells. The in planta functionality of FLP/FRT system has been previously demonstrated in Arabidopsis for excisional recombination (Luo et al., 2000) and in rice. Therefore, a recombination system, such as the FLP/FRT recombination system, can be used to control, through hybridization to FLP-expressing plants, the down-regulation of a plant flowering promotion gene, such as a FLO/LFY homolog, or up-regulation of a plant flowering repressor gene, producing controlled vegetative growth of transgenic plants.

A particular example of using site-specific DNA recombination to reduce transgene escape includes the following. A first fertile plant having one or more desirable traits is crossed with second fertile plant. The first or second plant, or both, can be transgenic. The second plant can also have one or more desirable traits. In one example, a transgene confers the desirable trait. The first fertile plant includes a first vector, wherein the first vector includes a promoter operably linked to a blocking sequence, and the blocking sequence is flanked by a recombining site sequence. The first vector also includes one or more nucleic acid sequences that reduce expression of a flower promotion gene, or one or more nucleic acid sequences that increase expression of a flower repressor gene sequence. Such nucleic acid sequences are downstream of the blocking sequence such that the nucleic acid sequence that reduces expression of a flower promotion gene or the nucleic acid sequence that increases expression of a flower repressor gene sequence is operably linked to the promoter upon recombination of the recombining site sequence

The second fertile plant includes a second vector which includes a recombinase, such as a promoter operably linked to a recombinase. In particular examples, the recombinase is integrated in the genome of the second fertile plant. The method includes permitting expression of the recombinase in the second fertile plant, or permitting expression of the recombinase in the resulting hybrid progeny of the first and second fertile plants. Expression of the recombinase will remove the blocking sequence from the first vector, resulting in the promoter being operably linked to the nucleic acid sequence that reduces expression of a flower promotion gene or the nucleic acid sequence that increases expression of a flower repressor gene sequence. Expression of the nucleic acid sequence that reduces expression of a flower promotion gene or the nucleic acid sequence that increases expression of a flower repressor gene sequence results in production of a transgenic plant with total vegetative growth, thereby reducing transgene escape by the transgenic plant. The second vector can further include a promoter operably linked to a selectable marker.

The promoter operably linked to the recombinase can be a constitutive promoter, such as a ubiquitin promoter, for example a rice ubiquitin promoter. In other examples, the promoter operably linked to the recombinase is an inducible promoter, and permitting expression of the recombinase includes contacting the second fertile plant with an inducing agent (thereby activating the inducible promoter). Exemplary inducible promoters include, but are not limited to, a heat shock promoter, a chemically inducible promoter, or a light activated promoter. The inducing agent (such as heat, a chemical, or light) can be contacted with the second fertile plant before or during crossing with the first fertile plant, or can be contacted with the resulting hybrid progeny following the crossing.

Exemplary recombinases and recombining sites include, but are not limited to: FLP/FRT, CRE/lox, RIRS sequence, and Gin/gix. Blocking sequences are known in the art, and include selectable marker gene sequences, such as a hyg, or bar, or pat cDNA sequence.

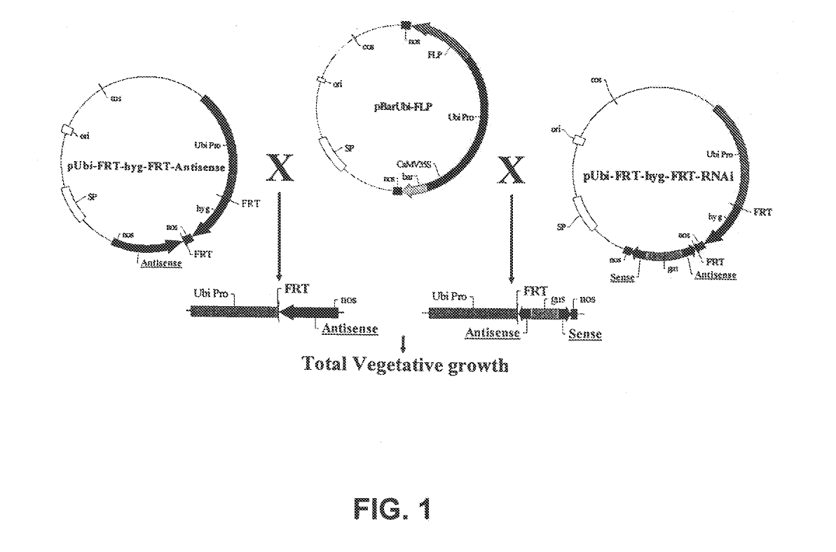

In a specific examples, controlled vegetative growth in transgenic turfgrass is achieved using the following method. Plants containing a vector in which the rice ubiquitin promoter and an RNAi or antisense molecule specific for a turfgrass FLO/LFY homolog is separated by the hyg gene flanked by directly oriented FRT sites will flower normally to produce seeds. When crossed to a plant expressing FLP recombinase, FLP will excise the blocking fragment (hyg gene) thus bringing together the ubiquitin promoter and the downstream RNAi or antisense molecule specific for the turfgrass FLO/LFY homolog, resulting in down-regulation of the FLO/LFY homolog gene and total vegetative growth in the hybrid (FIG. 1).

### Vectors

Provided by the present disclosure are vectors which can be used to practice the methods disclosed herein. Such vectors can be used to generate transgenic plants, such as plants that have decreased trangene escape and in some examples increased biomass production.

In one example, a vector includes a promoter operably linked to a blocking sequence, wherein the blocking sequence is flanked by a recombining site sequence. An example of a blocking sequence is a cDNA encoding a selectable marker (or a variant or fragment thereof that retains at least 50% of the desired biological activity), such as a hyg, or bar, or pat gene sequence. Exemplary recombining site sequences include, but are not limited to, an FRT sequence, a lox sequence, an RS sequence, or a gix sequence. The vector also includes a nucleic acid sequence that reduces expression of a flower promotion gene, such as an antisense or RNAi that specifically recognizes a flower promotion gene, downstream of the blocking sequence such that the nucleic acid sequence that reduces expression of a flower promotion gene is operably linked to the promoter upon recombination of the recombining site sequence. Alternatively, the vector also includes a nucleic acid sequence that increases expression of a flower repressor gene sequence, downstream of the blocking sequence such that the nucleic acid sequence that increases expression of a flower repressor gene sequence, is operably linked to the promoter upon recombination of the recombining site sequence.

The vector can further include a second promoter operably linked to a recombinase. In particular examples, the second promoter operably linked to the recombinase is an inducible promoter and the first promoter operably linked to the blocking sequence is a constitutive promoter.

### Example 1

### Production of Transgenic Bentgrass Expressing Yeast Recombinase FLP

This example describes methods used to generate a transgenic bentgrass expressing recombinase FLP.

Briefly, the vector pBarUbi-FLP (FIG. 1), which contains the corn ubiquitin promoter (Ubi Pro) promoting expression of the yeast FLP recombinase (FLP) and a CaMV 35S promoter promoting expression of the herbicide resistance gene bar, was synthesized to transform bentgrass using Agrobacterium-mediated plant transformation to produce transgenic plants expressing recombinase FLP.

Mature seeds were surface sterilized in 10% (v/v) Clorox® bleach plus two drops of Tween-20™ (Polysorbate 20) with vigorous shaking for 90 min. After rinsing five times in sterile distilled water, the seeds were placed onto callus-induction medium containing MS basal salts and vitamins (Murashige and Skoog 1962), 30 g/l sucrose, 500 mg/l casein hydrolysate, 6.6 mg/l 3,6-dichloro-o-anisic acid (dicamba), 0.5 mg/l 6-benzylaminopurine (BAP) and 2 g/l Phytagel. The pH of the medium was adjusted to 5.7 before autoclaving at 120° C. for 20 min. The culture plates containing prepared seed explants were kept in the dark at room temperature for 6 weeks. Embryogenic calli were visually selected and subcultured on fresh callus-induction medium in the dark at room temperature for 1 week before co-cultivation.

The transformation process can be divided into 5 sequential steps: agro-infection, co-cultivation, antibiotic treatment, selection, and plant regeneration. One day before agro-infection the embryogenic callus was divided into 1-2 mm pieces and placed on callus-induction medium containing 100 μM acetosyringone. Ten μl of Agrobacterium suspension (OD=1.0 at 660 nm) was then applied on each piece of callus, followed by 3 days of co-cultivation in the dark at 25° C. For the antibiotic treatment step, the callus was then transferred and cultured for 2 weeks on callus-induction medium plus 125 mg/l cefotaxime and 250 mg/l carbenicillin to suppress bacterial growth; and then, for selection, moved to callus-induction medium containing 250 mg/l cefotaxime and 10 mg/l phosphinothricin (PPT) for 8 weeks.

Antibiotic treatment and the entire selection process were performed at room temperature in the dark. The subculture interval during selection was typically 3 weeks. For plant regeneration, the PPT- or hygromycin-resistant proliferating callus was first moved to regeneration medium (MS basal medium, 30 g/l sucrose, 100 mg/l myo-inositol, 1 mg/l BAP and 2 g/l Phytagel) supplemented with cefotaxime, PPT or hygromycin. These calli were kept in the dark at room temperature for one week and then moved into the light for 2-3 weeks to develop shoots. Small shoots were then separated and transferred to hormone-free regeneration medium containing PPT or hygromycin cefotaxime to promote root growth while maintaining selection pressure and suppressing any remaining Agrobacterium cells. Plantlets with well-developed roots (3-5 weeks) were then transferred to soil and grown either in the greenhouse or in the field. Transient assay by bombardment of leaves from the transgenic FLP-containing plants with a FRT recombination-GUS reporter gene construct indicated the expression and function of the FLP recombinase in transformed turfgrass.

### Example 2

### Cloning of Bentgrass FLO/LFY Homolog Sequence

This example describes methods used to amplify a 250-bp DNA fragment, corresponding to the 3′-end of the bentgrass FLO/LFY homolog.

The primers 5′-CTACATCAACAAGCCCAAGATGCG-3′ (SEQ ID NO: 2) and 5′-CCTGGTGGCAGAGCTGGC-3′ (SEQ ID NO: 3) were used to PCR amplify a 250-bp DNA fragment from Agrostis stolonifera L. (SEQ ID NO: 1), corresponding to the 3′-end of the bentgrass FLO/LFY homolog.

The amplified fragment from bentgrass has been cloned into the EcoRI-BamHI sites of pLitmus28 (Biolabs).

Southern blot analysis of bentgrass genomic DNA (10 μg) isolated from leaves and digested with PstI, Hind III, or EcoRI using the amplified PCR fragment as a probe (SEQ ID NO: 1) revealed that the FLO/LFY homolog gene is present as a single copy in bentgrass genome.

Temporal and spatial expression of this FLO/LFY homolog was been examined by Northern hybridization analysis. Total RNA (20 μg) from leaves, roots, and the whole inflorescences of bentgrass were probed with the PCR product generated above (SEQ ID NO: 1). The transcript (≈1200 nt) was detected only in flowers, not in leaves or roots.

RNAi and antisense sequences RNAi and antisense constructs can be generated using the whole DNA sequence shown in SEQ ID NO: 1.

### Example 3

### Generation of Vectors

This example describes methods that can be used to generate two pSB 11-based Agrobacterium binary vectors (Komari et al., 1996). The vectors include a bentgrass FLO/LFY homolog sequence, an antisense of the turfgrass FLOA/LFY homolog, or an RNAi of the turfgrass FLOA/LFY homolog, under the control of a rice ubiquitin promoter. One skilled in the art will appreciate that similar methods can be used to generate similar vectors with other flower-related genes, for example by substituting the antisense or RNAi sequence of another flowering promotion gene, for the antisense or RNAi sequence of bentgrass FLO/LFY homolog. In addition, one skilled in the art will understand that other promoters can be used, and that the other recombinase systems can be used in place of the FLP/FRT system, such as the Cre/lox system.

Using an isolated turfgrass flower-related gene described in Example 2, pSB 11-based Agrobacterium binary vectors (Komari et al., 1996) for transformation of turfgrass with the chimeric gene construct consisting of either a RNAi construction using the bentgrass FLO/LFY homolog, or an antisense of the turfgrass FLO/LFY homolog under the control of a rice ubiquitin promoter can be generated.

In order to demonstrate the efficacy of antisense and RNAi technologies in reducing expression of a bentgrass FLO/LFY homolog for total vegetative growth of transgenic bentgrass, two gene constructs are generated which include the rice ubiquitin promoter to drive expression of the RNAi construct or the antisense sequence of the turfgrass FLO/LFY homolog. Both constructs can include a CaMV35S promoter to drive expression of a hygromycin resistance gene (hyg) as selectable marker for plant transformation.

To synthesize the antisense of the turfgrass flower-related gene-containing vector, p35S-hyg-Ubi-Antisense, the cloned C-terminal region of the turfgrass FLO/LFY homolog is released from the pAsLFY vector by BamHI-SnaBI digestions and ligated, in reverse orientation (antisense), into the BamHI-SacI (blunt-ended by Mung bean nuclease treatment) sites of pSBUbi-gus containing a rice ubiquitin promoter-driving gus gene, replacing gus coding region and giving rise to pSBUbi-Antisense. The Ubi-Antisense fragment can then be released by EcoRI digestion and ligated into the corresponding site of a binary vector, pSB35S-hyg resulting in p35S-hyg-Ubi-Antisense.

To synthesize the RNAi vector of the bentgrass flower-related gene, p35S-hyg-Ubi-RNAi, for expressing dsRNA in plant cells, the 35S-hyg fragment is released from pSB35S-hyg through HindIII digestion, and cloned into the corresponding site of the binary vector pSBUbi-gus, resulting in p35S-hyg-Ubi-gus. This vector is used as a bridge vector, in which an 824 by fragment of gus gene encoding β-glucuronidase is placed in between the rice ubiquitin promoter and the nopaline synthase (nos) terminator. The cloned C-terminal region of the turfgrass FLO/LFY homolog (SEQ ID NO: 1) will be released from pAsLFY by StuI-SnaBI digestions and placed, upstream (SmaI site) and downstream (flushed Sad site) of the gus fragment in opposite directions, in p35S-hyg-Ubi-gus, resulting in p35S-hyg-Ubi-gus-RNAi. The gus fragment is used as a linker between gene-specific fragments in the antisense and sense orientations.

### Example 4

### Generation of Vectors for Site-Specific Recombination

This example describes methods that can be used to synthesize two vectors, similar to that described in Example 3, except that the RNAi or the antisense of the turfgrass FLO/LFY homolog is separated from the ubiquitin promoter by the hygromycin-resistant gene, hyg that is flanked by FLP site-specific recombination target sites, FRTs.

In order to obtain transgenic turfgrass plants whose total vegetative growth are controlled by FLP/FRT site-specific recombination, two vectors are prepared in which the rice ubiquitin promoter and the RNAi construct or the antisense of the turfgrass FLO/LFY homolog is separated by the hyg gene flanked by directly-oriented FRT sites.

To synthesize the antisense of the turfgrass flower-specific gene-containing construct, pUbi-FRT-hyg-FRT-Antisense, the cloned C-terminal region of the turfgrass FLO/LFY homolog will be released from the pAsLFY plasmid by StuI-SnaBI digestions and ligated, into the KpnI-SacI (blunt-ended by Mung bean nuclease treatment) of the binary vector, pSBUbi-FRT-hyg-FRT-gus to replace the gus coding region. The orientation of the turfgrass FLO/LFY homolog gene inserted by blunt-end ligation will be checked by sequencing and the clone with the turfgrass FLO/LFY homolog in reverse orientation (antisense), pUbi-FRT-hyg-FRT-Antisense (FIG. 1), will be retained for further use.

To synthesize the RNAi construct of the turfgrass flower-specific gene, pUbi-FRT-hyg-FRT-RNAi, for expressing dsRNA in plant cells, we will first use pSBUbi-gus as a bridge vector, in which an 824 by fragment of gus gene encoding β-glucuronidase is placed in between the rice ubiquitin promoter and the nopaline synthase (nos) terminator. The cloned C-terminal region of the turfgrass FLO/LFY homolog will be released from pAsLFY by StuI-SnaBI digestions and placed, upstream (SmaI site) and downstream (flushed Sad site) of the gus fragment in opposite directions, in pSBUbi-gus, producing pUbi-gus-RNAi. Here, the gus fragment is used as a linker between gene-specific fragments in the antisense and sense orientations. The blocking DNA fragment, FRT-flanked hyg gene plus the nos terminator, FRT-hyg-FRT, will be released from pSBUbi-FRT-hyg-FRT-Gus as a SnaBI-KpnI fragment and ligated into the BamHI (flushed)-KpnI sites of the pSBUbi-gus-RNAi plasmid between the rice ubiquitin promoter and the downstream RNAi construction, giving rise to the final test vector pSBUbi-FRT-hyg-FRT-RNAi.

### Example 5

### Production of Transgenic Bentgrass

This example describes methods that can be used to generate transgenic bentgrass lines that include the vectors generated in Examples 3 and 4. Although this example describes use of Agrobacterium-mediated transformation, one skilled in the art will appreciate that other transformation methods can be used.

The four constructs described in Examples 3 and 4 are separately introduced into Agrobacterium tumefaciens LBA4404 by triparental mating or electroporation (Hiei et al., 1994). For triparental mating, the LBA4404 (pSB1) strain is grown on an AB+tetracycline (10 μg/ml) plate at 28° C. for 2-3 days. The E. coli strain, HB101 containing either of the four gene constructs described above is grown on a LA (LB agar medium)+spectinomycin (30 μg/ml) plate at 37° C. overnight. The conjugal helper E. coli strain, pRK2013 is also grown on a LA+kanamycin (50 mg/ml) at 37° C. overnight. One loopful each of the 3 strains is mixed onto a Nutrient Agar (Difco) plate and incubated at 28° C. overnight. The mixture is then streaked out onto an AB+spectinomycin (50 μg/ml) plate and incubated at 28° C. for 3 days. A single colony is selected, streaked out on the same medium, and incubated as above. The same procedure is repeated and prepare plasmid DNA from the resultant strain and verify, by restriction digestion, the expected co-integration of the gene constructs described above into the Agrobacterium plasmid. When introducing the gene constructs described above into Agrobacterium tumefaciens LBA4404 by electroporation, the DNA of the gene construct is electroporated into the strain LBA4404 (pSB1) using Gene Pulser Apparatus (Bio-Rad) using conditions recommended by the manufacturer.

Mature seeds of bentgrass Penn-A-4 will be surface sterilized in 10% (v/v) Clorox® bleach plus two drops of Tween-20™ (Polysorbate 20) with vigorous shaking for 90 min. After rinsing five times in sterile distilled water, the seeds will be placed onto callus-induction medium containing MS basal salts and vitamins (Murashige and Skoog 1962), 30 g/l sucrose, 500 mg/l casein hydrolysate, 6.6 mg/l 3,6-dichloro-o-anisic acid (dicamba), 0.5 mg/l 6-benzylaminopurine (BAP) and 2 g/l Phytagel. The pH of the medium will be adjusted to 5.7 before autoclaving at 120° C. for 20 min. The culture plates containing prepared seed explants will be kept in the dark at room temperature for 6 weeks. Embryogenic calli will be visually selected and subcultured on fresh callus-induction medium in the dark at room temperature for 1 week before co-cultivation.

The four constructs described in Examples 3 and 4 are transformed into bentgrass (Penn-A-4) by Agrobacterium-mediated transformation using embryogenic callus (Luo et al., 2004a, b; 2005a).

The regenerated plants will be transferred into soil and grown in the greenhouse. Molecular characterization of these T₀ transformants will be performed to demonstrate the presence and expression of the introduced foreign genes, and determine the copy number of transgene insertion. Southern blot analysis will be performed on the turf transformants. Genomic DNA will be obtained from leaves using procedure described in QIAamp Tissue Kit (QIAGEN, Inc., Chatsworth, Calif.) for Southern analysis using either, hyg gene as probes following standard molecular biology techniques (Sambrook et al., 1989).

Transgenic plants with single-copy transgene insertion will be selected and grown to maturity in the greenhouse to examine the flowering status. Total RNA from leaf tissues of positively identified transgenic plants will be isolated to determine mRNA accumulation in separate transformants, using the RNeasy Plant Total RNA Kit (QIAGEN Inc., Chatsworth, Calif.). Ten μg total RNA will be fractionated on agarose gels in denaturing conditions (7.5% formaldehyde) for Northern analysis (Sambrook et al. 1989).

Agrobacterium-mediated transformation should yield 100-150 independent transgenic events for each gene construct. The transgenics containing 35S-hyg-Ubi-Antisense and 35S-hyg-Ubi-RNAi vectors will be analyzed, for example using Northern, Southern, or Western analysis, to determine the efficacy of antisense and RNAi technologies in reducing the expression of bentgrass FLO/LFY homolog for total vegetative growth of transgenic bentgrass.

### Example 6

### Analysis of Suppressing or Reducing the Transition from Vegetative to Reproductive Growth in Transgenics

Transgenic bentgrass plants expressing RNAi or antisense of the bentgrass FLORICAULA/LEAFY homolog will be germinated at the same time and grown, in parallel with the non-transgenic wild-type plants, in the greenhouse under the same conditions. All the plants will then be vernalized. The plant morphology will be observed and recorded for all the vernalized plants. The number of days the wild-type plants take from seeding to flowering and that the transgenic plants take from seeding to flowering (if this occurs) will be recorded. Using this method, will permit identification of transgenic plants that do not flower, and consequently, no reproductive growth at all, and those that have delayed flowering. Similar methods can be used for any plant of interest.

### Example 7

### Cross-Pollination with FLP-Expressing Plants to Produce Hybrid with Total Vegetative Growth and Analysis of Efficacy of FLP-Mediated DNA Excision and the Efficiency of RNAi- and Antisense-Mediated Down Regulation of the Turfgrass Flower-Specific Gene, the FLO/LFY Homolog

This example describes methods that can be used to cross pollinate transgenic plants to generate a hybrid plant that has total vegetative growth.

Transgenic plants containing the RNAi construction or the antisense of the bentgrass FLORICAULA/LEAFY homolog separated from the ubiquitin promoter by the hyg gene that is flanked by FRT sites, are cross-pollinated with pollen from the recombinase FLP-expressing plants generated in Example 2. Methods of cross-pollination are known (for example see Luo et al. 2004a). Since the antisense or RNAi-containing T₀ plants are hemizygous with respect to transgene inserted, only 50% of the hybrids will contain the transgenes. Transgenic hybrid plants can be identified using PCR to verify the presence of the rice ubiquitin promoter. These plants are then grown in the greenhouse and vernalized.

Expression of the FLP recombinase in these resulting hybrid plants will remove the blocking fragment (hyg gene), bringing together the ubiquitin promoter and the downstream RNAi construct or the antisense of the bentgrass FLO/LFY homolog gene. This will result in decreased expression of the FLO/LFY homolog, giving rise to a total vegetative growth in the hybrid. Decreased gene expression can be determined using any method in the art, such as Northern or Western analysis. The total vegetative growth of these hybrid plants will be examined in comparison with wild-type plants. Southern analysis will be conducted to check the occurrence of FLP-mediated excisional DNA recombination, and Northern analysis will be performed with RNA from inflorescences to check the down-regulation of the FLOLFY homolog gene.

Based on the results obtained, the transgenic lines that have shown total vegetative growth for producing homozygous plants are selected. These plants will then be used to test the effectiveness of the total vegetative growth in controlling transgene escape from genetically modified grass.

### Example 8

### Efficacy of Prevention of Transgene Escape

### Cage Method

This example describes methods that can be used to demonstrate the effectiveness and efficacy of the total vegetative growth, for example as compared to male sterility, for mitigation of transgene flow.

Briefly, caged cross-pollination studies can be used with the verified transgenic plants with total vegetative growth described in Example 7. For comparison, male-sterile and fertile transgenic plants (Luo et al., 2004b) can be included as controls to evaluate the efficiencies of male sterility and total vegetative growth for prevention of transgene escape.

Since creeping bentgrass is an out-crossing, wind pollinated, perennial species, two different methods can be used. In the first, 20 transgenic plants with total vegetative growth, or male-sterile plants, are arranged to grow next to each other in a cage built with Monofilament Polyester Environmental Microscreening 420 EX-61″(GreenThumb Group, Inc., Racine, Wis.). Twenty non-transgenic wild type bentgrass plants (cv. Penn-A-4) are also grown together in a separate cage as a positive control. Upon flowering and maturation, the inflorescences, if any, are harvested and dried. Seeds from each plant, if any, are germinated to obtain seedlings whose number will be counted.

In the second, 20 smaller cages are prepared, and in each of them, one transgenic plant with total vegetative growth, or male sterility are grown together with a non-transgenic, wild type plant. Upon flowering and maturation, the inflorescences from the non-transgenic plants will be harvested and dried. Seeds from each plant, if any, will be germinated to obtain seedlings whose numbers will be counted.

The data obtained will be used for statistic analysis, and a F-test will be used to test the null hypotesis to determine the effectiveness and the efficacy of total vegetative growth and male sterility in mitigating transgene flow in creeping bentgrass.

Since no flower or no pollen would be produced in plants with total vegetative growth or male sterility, no seed production should be observed in the plants with total vegetative growth, or male sterility, which are arranged to grow together in a cage. Similarly, when plants with total vegetative growth, or male sterility are arranged to grow together with non-transgenic, wild-type plants in a cage, plants with total vegetative growth cannot be pollinated with pollen from non-transgenic wild-type plant to produce viable seeds, and no seed production is expected in the wild-type plant either due to the failure of pollen production in the plants with total vegetative growth.

On the other hand, while the male-sterile plants could be pollinated with pollen from non-transgenic plant to produce viable seeds, no seed production would be expected in the wild-type plant due to the failure of viable pollen production in the male-sterile plant.

### Example 9

### Efficacy of Prevention of Transgene Escape

### Field Trail

This example describes methods that can be used to demonstrate the effectiveness and efficacy of the total vegetative growth, for example as compared to male sterility, for mitigation of transgene flow.

Briefly, a field trail study for gene flow using the verified transgenic plants with total vegetative growth (see Example 7), under isolated conditions. Approximately 350 non-transgenic bentgrasses cv. Penn-A-4 will be planted in transects around a 35×130 ft nursery (≈0.1 ac or 0.04 ha) containing approximately 300 transgenic plants. A similar ratio of transgenic to non-transgenic plants has been used and shown to be sufficient to detect the spread of the bar gene (Wipff and Fricker, 2001; 2000). Therefore, the size of this plot should provide sufficient pollen load to evaluate pollen dispersal and capability for intraspecifc gene flow. The design can be patterned after Wipff and Fricker (2001; 2000) where the following transects will be constructed as follows: 1) two circles around the nursery at 110 (33.5 m) and 275 ft (83.8 m) with plants spaced at 50 ft (15.24 m) and 100 ft (30.48 m), respectively; 2) two line-transects aligned with prevailing winds (NE) with one transect NE and another SW of the transgenic nursery, and two additional line-transects (SE and NW), orthogonal to the prevailing winds. The NE transect extends 245 ft (74.6 m) from the NE edge of the nursery and the SW, SE and NW transects extend 300 ft (91.4 m) from the SW, SE and NW edges of the nursery, respectively. Plants in the line-transects will be spaced 10 ft (3.048 m) apart for the first 120 ft (36.6 m), and then spaced 20 ft (6.1 m) after.

Once the plants have finished flowering, inflorescences of the non-transgenic plants will be enclosed in hybridization bags. Any remaining, un-bagged, inflorescences are cut and burned to prevent any contamination. The inflorescences are then harvested and the non-transgenic plants are killed with herbicide Roundup® and burned. The harvested inflorescences are dried in the greenhouse. Once dry, seeds from each non-transgenic plant will be planted and screened in greenhouse for herbicide resistance; around 1000 seeds will be planted in order to obtain 1,000 seedlings to be screened. The seedlings will then be sprayed 2 to 3 times with herbicide Finale™ once they reach the 3 to 4 leaf stage, with a rate of 5.7 L/0.4 ha (6 qts/ac). This rate has been tested on five different non-transgenic genotypes of creeping bentgrass (a total of 14,000,000 seedlings) and one genotype of colonial bentgrass (2,000,000 seedlings). No survivors were found, whereas the transgenic control bentgrass plants containing herbicide-resistant gene bar were not damaged with this rate.

Using this method, seedlings (if any) produced through transgene flow are recovered for subsequent molecular verification to confirm the presence of the bar gene using PCR and Southern blot analysis. The percentage of resistant seedling progeny will be calculated as the number of survivors divided by the total number of seedlings germinated. The data can be analyzed with Graphpad Prism® non-linear regression software. The curve that best fit the data was a ‘Top to Zero One Phase Exponential Decay’ Model. Since the goal of regression is to find a curve that best predict Y from X, an exponential decay model should fit the data well (Wipff and Fricker, 2001; 2000). This allows for the prediction of the percent recovery of the transgene over distance.

These results, together with that obtained from “pollen cage” study will demonstrate the completeness of total vegetative growth in the transgenic turfgrass expressing either the antisense or RNAi of the flower-specific gene, and the feasibility of using total vegetative growth as a tool in the prevention of gene flow. These methods can also provide information on how efficient the engineered total vegetative growth and male sterility in mitigation of transgene escape from the genetically modified grasses.

### REFERENCES CITED

1. Altieri (2000) Ecosystem Health 6:13-23.
2. Bomblies et al. (2003) Development, 130(11):2385-95.
3. Bradley et al. (1996) Nature 376:791-797.
4. Coen et al. (1990) Cell 63:1311-1322.
5. Dale (1992) Plant Physiol. 100:13-5.
6. Dale (1993) J Agr Sci 120:1-5.
7. Dale et al. (2002) Nat. Biotechnol. 20:567-74.
8. Daniell (2002) Nat. Biotechnol. 20:581-6.
9. De Block et al. (1997) Theor. Appl. Genet. 95:125-31.
10. Eastham and Sweet (2002) Genetically modified organisms (GMOs): the significance of gene flow through pollen transfer. European Environment Agency, Denmark.
11. Ellatrand and Hoffman (1990) Bioscience 40:438-42.
12. Ellstrand et al. (1999). Annu. Rev. Ecol. Syst. 30:539-63.
13. Goetz et al. (2001) Proc. Natl. Acad. Sci. USA 98:6522-7.
14. Hiei Y, Ohta S, Komari T, Kumashiro T (1994) Plant J 6:271-282.
15. Hoffman (1990) BioScience 40:434-7.
16. Jagannath et al. (2001) Mol Breed 8:11-23.
17. Johnson and Riordan (1999) Hort. Science 34:594-8.
18. Kyozuka et al. (1998) Proc. Natl. Acad. Sci. USA, 95:1979-82.
19. Luo et al. (2005a) Agrobacterium tumefaciens-mediated bentgrass transformation. In: Methods in Molecular Biology, Vol 44, Wang K (ed.), Humana Press Inc., Totowa, N.J. (in press).
20. Luo et al. (2004a) Controlling transgene escape in genetically modified grasses. In: Molecular Breeding of Forage and Turf, Hopkins A, Wang Z Y, Mian R, Sledge M and Barker R (eds.), Kluwer Academic Publishers, Dordrecht/Boston/London, p 245-254.
21. Luo et al. (2004b) Plant Cell Rep. 22:645-52.
22. Luo et al. (2000) Plant J. 23:423-30.
23. Lyznik et al. (1993) Nucleic Acids Res 21:969-75.
24. Mariani et al. (1990) Nature 347:737-41.
25. Michaels et al. (1999) Plant Cell 11:949-956.
26. Moffatt and Somerville (1988) Plant Physiol. 86:1150-4.
27. O'Gorman et al. (1991) Science 251:1351-5.
28. Rogers and Parkes (1995) J. Exp. Bot. 46:467-488.
29. Samach et al. (2000) Science 288:1613-6.
30. Shannon and Meeks-Wagner (1991) Plant Cell 3:877-92.
31. Simpson and Dean (2002) Science 296:285-9.
32. Sung et al. (1992) Science 258:1645-7.
33. Tsuchiya et al. (1995). Plant Cell Physiol 36:487-94.
34. Watrud et al. (2004) Proc. Nat. Acad. Sci. USA 101:14533-8.
35. Weigel et al. (1992) Cell 69:843-859.
36. Wipff and Fricker (2000) Diversity 16:36-9.
37. Wipff and Fricker (2001) Int. Turfgrass Soc. Res. J. 9:224-42.
38. Xu et al. (1995) Proc. Nat. Acad. Sci. USA 92:2106-10.
39. Zik and Irish (2003) Annu. Rev. Cell Dev. Biol. 19:119-40.
   All references cited herein are incorporated by reference.

In view of the many possible embodiments to which the principles of the disclosed invention may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

We claim:

1. A method of reducing transgene escape from a transgenic plant, comprising:
stably transforming a plant with a vector, wherein the vector comprises a sequence that reduces expression of a flower promotion gene, or a sequence that increases expression of a flower repressor gene, operably linked to a promoter, thereby producing a transgenic plant having total vegetative growth, and thereby reducing transgene escape from the transgenic plant.

2. The method of claim 1, wherein the method further enhances biomass production of the transgenic plant.

3. The method of claim 1, wherein the plant is a perennial.

4. The method of claim 3, wherein the plant is a turfgrass or a bentgrass (Agrostis stolonifera L.).

5. The method of claim 1, wherein the method produces total sterility in the transgenic plant.

6. The method of claim 1, wherein the flower promotion gene is a floral meristem identity gene.

7. The method of claim 1, wherein transgene escape is reduced by at least 95%, relative to a plant not transformed with the vector.

8. The method of claim 1, further comprising selecting stably transformed plants having reduced transgene escape.

9. The method of claim 1, wherein the reduced transgene escape is maintained through vegetative propagation of the plant.

10. The method of claim 2, wherein enhanced biomass production is maintained through vegetative propagation of the plant.

11. A plant produced by the method of claim 1.

12. Seed of the plant of claim 11.

13. A method of reducing transgene escape from a transgenic plant, comprising:
stably transforming a plant with a vector, wherein the vector comprises an inducible promoter operably linked to a nucleic acid sequence that encodes a FLP recombinase, a promoter operably linked to a nucleic acid sequence that encodes a selectable marker, wherein the nucleic acid sequence that encodes a selectable marker is flanked by a FRT recombining site, and a nucleic acid sequence that reduces expression of a flower promotion gene downstream of the nucleic acid sequence that encodes a selectable marker such that the nucleic acid sequence that reduces expression of a flower promotion gene is operably linked to the promoter upon recombination of the FRT recombining site sequence,
exposing the plant to an inducing agent to permit expression of the recombinase, thereby permitting expression of the sequence that reduces expression of the flower promotion gene, thereby producing a transgenic plant having total vegetative growth, and thereby reducing transgene escape from the transgenic plant.

14. A vector comprising:
a rice ubiquitin promoter operably linked to a nucleic acid sequence that encodes a selectable marker, wherein the nucleic acid sequence that encodes a selectable marker is flanked by a FRT recombining site sequence;
a nucleic acid sequence that reduces expression of a flower promotion gene downstream of the nucleic acid sequence that encodes a selectable marker such that the nucleic acid sequence that reduces expression of a flower promotion gene is operably linked to the rice ubiquitin promoter upon recombination of the FRT recombining site sequence wherein upon expression in a transgenic plant the transgenic plant will have total vegetative growth, and thereby reduce transgene escape from the transgenic plant.

15. The vector of claim 14, wherein the nucleic acid sequence that encodes a selectable marker comprises a hyg, or bar, or pat gene sequence.

16. The vector of claim 14, further comprising a promoter operably linked to a recombinase.

17. The vector of claim 16, wherein the promoter operably linked to the recombinase is an inducible promoter and the promoter operably linked to the blocking sequence is a constitutive promoter.

18. A method of reducing transgene escape from a transgenic plant, comprising:
crossing a first fertile transgenic plant having a desirable trait with second fertile plant, wherein the first fertile transgenic plant comprises the vector of claim 14, and wherein the second fertile plant comprises a second vector comprising a promoter operably linked to a nucleic acid sequence that encodes a FLP recombinase; and
permitting expression of the FLP recombinase, wherein crossing the first and second fertile plant results in production of a hybrid plant with total vegetative growth, thereby reducing transgene escape from the transgenic plant.

19. The method of claim 18, wherein the promoter operably linked to the nucleic acid sequence that encodes the FLP recombinase is a constitutive promoter.

20. The method of claim 18, wherein the promoter operably linked to the nucleic acid sequence that encodes the FLP recombinase is an inducible promoter, and wherein permitting expression of the nucleic acid sequence that encodes the FLP recombinase comprises contacting the second fertile plant with an inducing agent.

21. The method of claim 18, wherein the second vector further comprises a promoter operably linked to a selectable marker.

22. The method of claim 18, wherein the blocking sequence is a selectable marker gene sequence.

23. The method of claim 18, wherein the flower promotion gene is a floral meristem identity gene involved in the transition from vegetative to reproductive development in plant.

24. The method of claim 18, wherein the nucleic acid sequence that encodes the FLP recombinase is integrated in the genome of the second fertile plant.

25. A plant produced by the method of claim 18, wherein the plant has enhanced biomass production.

26. Seed of the plant of claim 25.

27. The plant according to claim 25, wherein the plant is a bentgrass plant.
